(19) United States
(12) Patent Application Publication
(10) Pub. No.: US 2014/0228724 A1
(43) Pub. Date: Aug. 14, 2014

(54) PROTECTIVE COVER

(71) Applicant: Robert Casey, Bristol, CT (US)

(72) Inventor: Robert Casey, Bristol, CT (US)

(21) Appl. No.: 14/181,125

(22) Filed: Feb. 14, 2014

Related U.S. Application Data
(60) Provisional application No. 61/764,867, filed on Feb. 14, 2013.

(51) Int. Cl. A61F 5/01

(52) U.S. Cl. CPC A61F 5/01
USPC 602/3

(57) ABSTRACT

Disclosed is a protective cover configured for covering a limb, the protective cover including an upper portion formed of a first non-rigid material; and a lower portion formed of at least one semi-rigid second material.

Further disclosed is a protective cover configured for covering a limb, the protective cover including an upper portion formed of a first non-rigid material; and a lower portion formed of said first non-rigid material and at least one second semi-rigid material.

### CROSS REFERENCE TO RELATED APPLICATIONS

This application claims the benefit of U.S. Provisional Patent Application 61/764,867 filed on Feb. 14, 2013, of which said application is herein incorporated by reference in its entirety.

### FIELD

The disclosure relates generally to a protective cover, and more particularly to a protective cover for animals.

### BACKGROUND

Conventional casts or bandages applied to the limb of an animal, such as a dog, may be damaged or otherwise negatively affected by moisture and dirt, requiring the use of a protective cover to keep the underlying cast or bandage clean and dry while the wearer continues to walk or otherwise place the limb on the ground during recovery. Such covers may lose retention, slip off, and further become damaged during use, allowing for moisture and contaminants to damage the underlying cast or bandage.

Accordingly, a protective cover that is capable of adequately covering a cast or bandage while withstanding typical wear during an animal's normal activities would be desirable.

### SUMMARY

Disclosed is a protective cover configured for covering a limb, the protective cover including an upper portion formed of a first non-rigid material; and a lower portion formed of at least one semi-rigid second material.

Further disclosed is a protective cover configured for covering a limb, the protective cover including an upper portion formed of a first non-rigid material; and a lower portion formed of said first non-rigid material and at least one second semi-rigid material.

### BRIEF DESCRIPTION OF THE FIGURES

The accompanying drawings incorporated in and forming a part of the specification embodies several aspects of the present invention and, together with the description, serve to explain the principles of the invention. In the drawings:

FIG. 1 is a perspective view of a protective cover in accordance with an exemplary embodiment;

FIG. 2 is a top plan view of the protective cover of FIG. 1; and

FIG. 3 is a schematic elevation view of a protective cover in accordance with an alternative exemplary embodiment.

### DETAILED DESCRIPTION

The following disclosure will detail particular embodiments according to the present invention, which provides a protective cover, and more particularly to a protective cover for animals. Reference is now made to the drawings, wherein like reference numerals are used to refer to like elements throughout the disclosure.

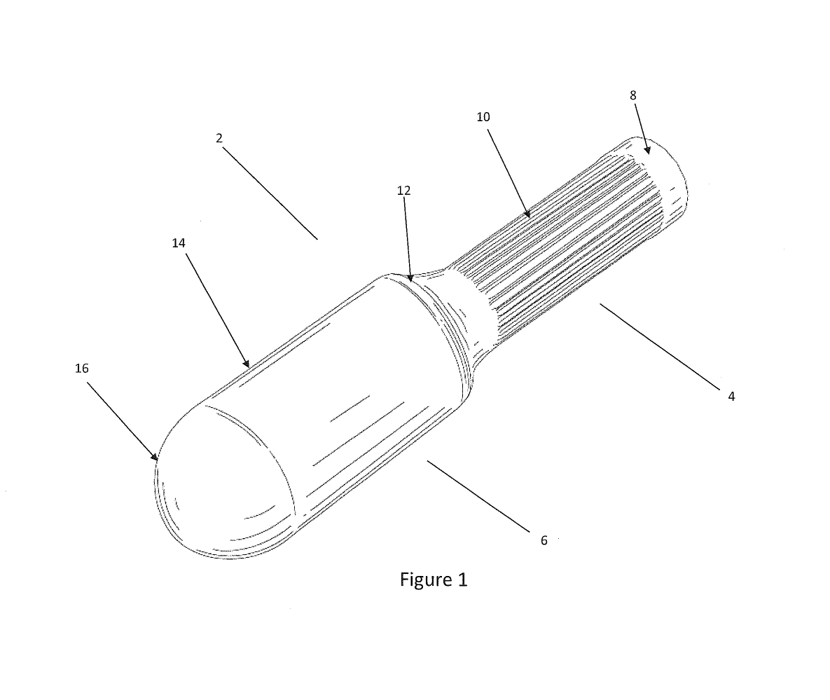

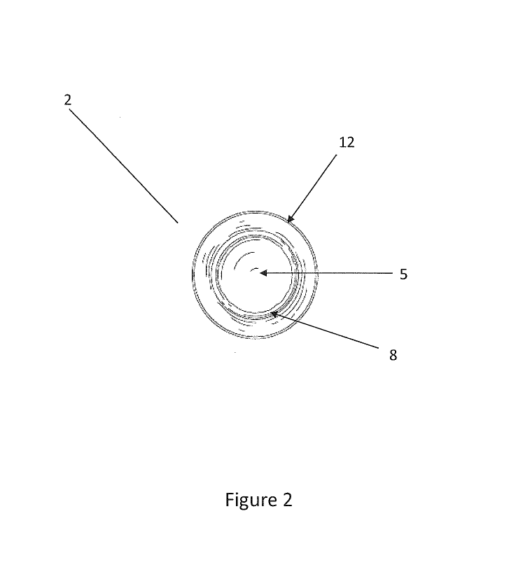

Referring to FIGS. 1 and 2, a protective cover 2 utilized to protect a bandage or cast and the limb of an animal is shown. Protective cover 2 includes an upper portion 4 and a semi-rigid lower portion 6 disposed below the upper portion 4.

The the upper portion 4 of protective cover 2 is formed of a non-rigid, conforming material. As defined herein, a non-rigid material will not retain a general shape when the material is not disposed around an object or otherwise shaped. In an exemplary embodiment, the upper portion 4 is formed of an elasticized fabric wherein the fabric can stretch, contract, and generally conform to the wearer's limb, cast, or bandage. Further, the fabric may expand and contract while being fitted or removed and additionally limit slipping during use. In at least one embodiment, the fabric may be cotton, synthetic fibers, synthetic blend, elastic, or a combination thereof.

Upper portion 4 may have a generally cylindrical shape with the upper extent having an opening 5 (shown in FIG. 2) to allow a limb to be inserted into the protective cover 2. In at least one embodiment, opening 5 may have an elastic band 8 circumferentially formed therein to secure the upper portion 4 and protective cover 2 generally to and around the wearer's limb to keep moisture and contaminants out. Further, elastic band 8 allows opening 5 to expand and contract during the application and removal of protective cover 2.

In an exemplary embodiment, the upper portion 4 is of any suitable length and diameter and is adapted to the animal's needs. In at least one embodiment, the upper portion 4 has elastic strips 10 of material longitudinally disposed to contain and fold excess material within upper portion 4 for when upper portion 4 is not fully expanded. Elastic strips 10 may then deploy needed material during expansion. Compared to prior art covers, the design of upper portion 4 and lower portion 6 eliminates any loose material that the animal can grab while ensuring a secure fit of the protective cover 2. Advantageously, the lack of loose material reduces snagging or tripping experienced by the wearer. Furthermore, noise while wearing protective cover 2 is eliminated.

Upper portion 4 has a lower extent including a transition area 12 to transition the upper portion 4 to the lower portion 6 of protective cover 2. The transition area 12 may allow for a smooth transition between the change in diameter of the upper portion 4 and the lower portion 6. The lower portion 6 may be larger due to the semi-rigid construction of lower portion 6, discussed below, causing a difference in diameters.

In an exemplary embodiment, lower portion 6 is formed of a semi-rigid, conforming material. As defined herein, a semi-rigid material retains its shape even when otherwise not shaped, while still allowing for elastic deformation. In at least one embodiment, the non-rigid material of the upper portion 4 additionally forms the first layer of the lower portion 6 wherein a semi-rigid material is applied over the non-rigid material.

In an exemplary embodiment, the semi-rigid material is a coating that is water resistant. In at least one embodiment, the semi-rigid material is rubber, latex rubber, polymer or any other suitable material. The semi-rigid material may enhance rigidity, durability, and grip of the protective cover 2. Further, the material of the lower portion 6 can withstand normal wear from the animal's activities and continue to keep water and contaminants away from the animal's limb, cast, or bandage. In an exemplary embodiment, the semi-rigid material is conforming and fits various sized limbs, casts or bandages.

The semi-rigid material may be sprayed, painted, dipped or otherwise applied. The coating is cured to a flexible finished state, wherein the shape during forming is retained. In an exemplary embodiment, the shape retained by lower portion 6 is adapted to conform to the animal's limb, bandaged limb, or cast. Multiple layers of the semi-rigid material may be applied to increase the thickness of the material and increase the durability of the protective cover 2.

In at least one embodiment, the semi-rigid material of the lower portion 6 is at least ⅓ to at least ½ of the entire length protective cover 2. Accordingly, since the semi-rigid material has water resistant properties, the lower portion 6 may provide moisture protection from the environment to the protected cast or bandage, or at least a portion of the protected cast or bandage. Compared to prior art coverings, the entire lower portion 6 may provide moisture protection beyond merely the lowermost extent of the lower portion 6. The length and characteristics of the semi-rigid material may allow for superior moisture protection from grass, water, and snow that may reach above traditional water proof coverings. In other embodiments, the non-rigid upper portion 4 is a greater proportion of the protective cover 2.

The lower portion 6 is configured to fit over a cast, soft cast, splinted and wrapped, or heavily bandaged limb. The characteristics of the material and the shape of lower portion 6 allow for a form fitting shape that is compliant. The lower portion 6, particularly the lower extent of lower portion 6 may have a rounded end 16 to conform to the end of the limb of the animal or the end of the bandage or cast to be covered while providing an external gripping surface for the animal.

In at least one embodiment, the protective cover 2, and the lower portion 6 particularly is formed straight, without any bends. In other words, lower portion 6, and protective cover 2 generally does not have a relative angle (or wrist angle) that follows the limb angles of an uninjured animal because most post-surgical limbs are fully wrapped in casting or bandaging with no need for a relative angle between the upper portion 4 and the lower portion 6. Compared to prior art covers that may have a molded or otherwise prescribed angles, the relation of the upper portion 4 and lower portion 6 does not interfere with a complete and proper fit to the straight geometry of the typical cast, soft cast, or splint with a heavy bandage.

Due to the length and rigidity of sleeve 14 of the lower portion 6 and the relative angle and conforming nature of protective cover 2, the protective cover 2 may be self-aligning relative to the upper portion 4 to ensure a fit while the animal is moving. The lower portion 4 may be pushed back into alignment or into a proper place when force is applied; compared to prior art covers where the cover may come off.

An appropriate size protective cover 2 is selected for the animal's leg to cover the casting or bandaging on the animal's leg. Protective cover 2 may be used for other applications, particularly other animal applications. The protective cover 2 is slipped on through the opening 5 in the upper portion 4 and pulled until the lower portion 6 is fully covering the limb. The length and diameter, and the length of the sleeve 14 and lower portion 6 in relation to the upper portion 4 can be varied based on application. The protective cover 2 may be removed and replaced as needed.

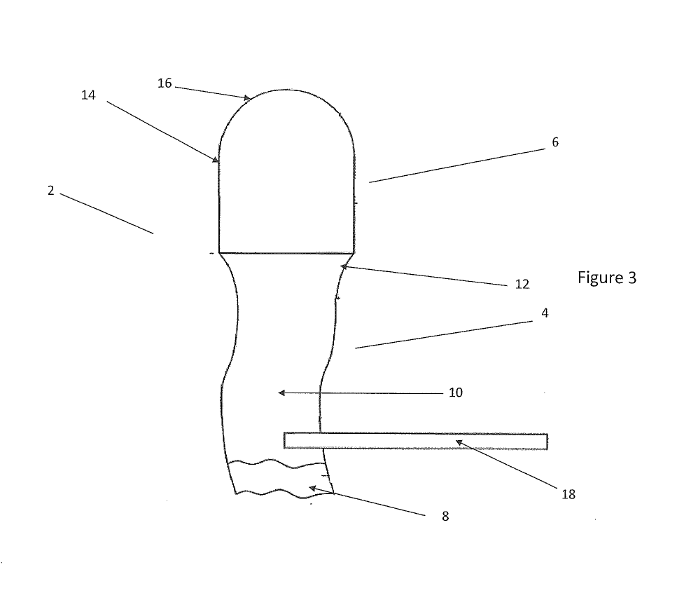

Referring to FIG. 3, an additional embodiment of protective cover 2 is shown. In this alternative embodiment, the upper portion 4 further includes hook and loop fastener 18 to work in conjunction with elastic band 8 and elastic strips 10 to secure upper portion 4 and protective cover 2 generally against the animal's limb. The hook and loop fastener 18 may be wrapped around the upper portion 4 and the animal's limb, bandage or cast to further secure upper portion 4 and protective cover 2 generally. The hook and loop fastener 18 may be affixed to upper portion 4 by sewing or heat activated adhesive.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Exemplary embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A protective cover adapted to cover a limb, the protective cover comprising:
an upper portion formed of a first non-rigid material; and
a lower portion disposed below said upper portion, said lower portion formed of at least one semi-rigid second material.

2. The protective cover of claim 1, wherein said first non-rigid material is cotton, synthetic, or synthetic blend.

3. The protective cover of claim 1, wherein said at least one second semi-rigid material is at least one of rubber, latex rubber, and polymer.

4. The protective cover of claim 1, wherein said at least one second semi-rigid material is water resistant.

5. The protective cover of claim 1, wherein said lower portion is adapted to conform to a limb of an animal.

6. The protective cover of claim 1, wherein said lower portion has a rounded lower extent.

7. The protective cover of claim 1, wherein said lower portion is formed absent any bends.

8. The protective cover of claim 1, wherein said lower portion is at least ⅓ the length of the protective cover.

9. The protective cover of claim 8, wherein said lower portion is at least ½ the length of the protective cover.

10. The protective cover of claim 1, wherein said lower portion is configured to provide a gripping surface.

11. The protective cover of claim 1, wherein said upper portion is adapted to conform to a limb of an animal.

12. The protective cover of claim 1, said upper portion further comprising a hook and loop fastener adapted to secure the protective cover to a limb of an animal.

13. The protective cover of claim 1, said upper portion further comprising an elastic band adapted to secure the protective cover to a limb of an animal.

14. The protective cover of claim 1, said upper portion further comprising at least one longitudinal elastic strip to gather excess material and adapted to secure said protective cover to a limb of an animal.

15. The protective cover of claim 1, wherein the protective cover is configured to be self-aligning.

16. A protective cover adapted to cover a limb, the protective cover comprising:
an upper portion formed of a first non-rigid material; and
a lower portion disposed below said upper portion, said lower portion formed of said first non-rigid material and at least one second semi-rigid material.

17. The protective cover of claim 16, wherein said first non-rigid material is cotton, synthetic, or synthetic blend.

18. The protective cover of claim 16, wherein said at least one second semi rigid material is disposed on top of said first material.

19. The protective cover of claim 16, wherein said at least one second semi-rigid material is at least one of rubber, latex rubber, and polymer.

20. The protective cover of claim 16, wherein said lower portion is adapted to conform to a limb of an animal.
